(19) United States
(12) Patent Application Publication
(10) Pub. No.: US 2023/0035339 A1
(43) Pub. Date: Feb. 2, 2023

(54) BIOMARKER COMPOSITION FOR DIAGNOSING PRE-ECLAMPSIA AND USE THEREOF

(71) Applicants: SUNGKWANG MEDICAL FOUNDATION, Seoul (KR); UNIVERSITY-INDUSTRY COOPERATION GROUP OF KYUNG HEE UNIVERSITY, Yongin-si (KR)

(72) Inventors: Hyun Mee RYU, Seongnam-si (KR); Ji Hyae LIM, Seongnam-si (KR); Kwang Pyo KIM, Seoul (KR)

(73) Assignees: SUNGKWANG MEDICAL FOUNDATION, Seoul (KR); UNIVERSITY-INDUSTRY COOPERATION GROUP OF KYUNG HEE UNIVERSITY, Yongin-si (KR)

(21) Appl. No.: 17/790,068

(22) PCT Filed: Dec. 28, 2020
(86) PCT No.: PCT/KR2020/019201

§ 371 Date: Jun. 29, 2022

(30) Foreign Application Priority Data
Dec. 31, 2019 (KR) 10-2019-0179979

(51) Int. Cl. G01N 33/68; G01N 33/563

(52) U.S. Cl. CPC G01N 33/689; G01N 33/563; G01N 2800/368

(57) ABSTRACT

Provided are: a composition for diagnosing pre-eclampsia; a kit; a method of diagnosing pre-eclampsia using the same; and a method of providing information on diagnosis of pre-eclampsia. The composition, the kit, and the methods provide the effect of enabling the accurate and sensitive diagnosis of pre-eclampsia in a subject.

### TECHNICAL FIELD

The application claims the benefit of Korean Patent Application No. 10-2019-0179979, the disclosure of which is incorporated herein in its entirety by reference.

The present disclosure relates to a biomarker composition for diagnosing pre-eclampsia and use thereof.

### BACKGROUND ART

Pre-eclampsia is a hypertensive (>140/90 mmHg) disease accompanied by proteinuria (>300 mg/day) that begins after 20 weeks of pregnancy due to various causes such as inflammation, hypoxia, and oxidative stress, and threatens the life of the pregnant women and the fetus. However, there is no specific treatment so far, and prevention is known to be the best way.

As a means for preventing and diagnosing pre-eclampsia, the PerkinElmer DELFIA® Xpress PIGF kit has been released. However, the kit shows a low detection rate of 44% at a false-positive rate of 5%, and thus is not useful in actual diagnosis, and the Alere Triage® PLGF test is also insufficient to prove the effectiveness in a clinical diagnosis.

With such a technical background, studies on an analysis method that can detect the disease in advance through genetic analysis for the above-mentioned disease are being actively conducted (Korean published patent 10-2019-0003987), but the output is still inadequate.

### DESCRIPTION OF EMBODIMENTS

### Technical Problem

As aspect is to provide a composition for diagnosing pre-eclampsia comprising: a formulation for measuring an expression level of at least one protein selected from the group consisting of ANXA3, A2M, APOB, PZP, FETUB, FNI, LCN2, APOM, QSOXI, TGOLN2, FIO, SERPINAII, PRG2, SHBG, TNC, HBD, AGT, CP, HEXB and SERPINA4, VWF, or a fragment thereof.

Another aspect is to provide a kit for diagnosing pre-eclampsia comprising the composition.

Still another aspect is to provide a method of providing information on diagnosis of pre-eclampsia, the method comprising: (a) measuring an expression level of at least one protein selected from the group consisting of ANXA3, A2M, APOB, PZP, FETUB, FNI, LCN2, APOM, QSOXI, TGOLN2, FIO, SERPINAII, PRG2, SHBG, TNC, HBD, AGT, CP, HEXB, SERPINA4, and VWF, or a fragment thereof in a biological sample isolated from a subject; (b) comparing the expression level of the measured protein or the fragment thereof with an expression level of the protein or the fragment thereof in a control group sample.

### Solution to Problem

An aspect provides a composition for diagnosing pre-eclampsia comprising a formulation for measuring an expression level of at least one protein selected from the group consisting of ANXA3, A2M, APOB, PZP, FETUB, FNI, LCN2, APOM, QSOXI, TGOLN2, FIO, SERPINAII, PRG2, SHBG, TNC, HBD, AGT, CP, HEXB, SERPINA4, and VWF, or a fragment thereof.

The ANXA3 is also known as Annexin A3. The A2M is also known as alpha-2-Macroglobulin or α2M. The APOB is also known as Apolipoprotein B. The PZP is also known as PZP alpha-2-macroglobulin like or pregnancy zone protein. The FETUB is also known as Fetuin B. The FN1 is also known as Fibronectin 1. The LCN2 is also known as Lipocalin 2, oncogene 24p3, or neutrophil gelatinase-associated lipocalin (NGAL). The APOM is also known as Apolipoprotein M. The QSOX1 is also known as Quiescin sulfhydryl oxidase 1 or Quiescin Q6. The TGOLN2 is also known as Trans-golgi network protein 2. The F10 is also known as coagulation factor X. The SERPINA11 is also known as Serpin Family A member 11. The PRG2 is also known as Proteoglycan 2, pro eosinophil major basic protein. The SHBG is also known as Sex hormone binding globulin. The TNC is also known as Tenascin C. The HBD is also known as Hemoglobin subunit delta. The AGT is also known as Angiotensinogen. The CP is also known as ceruloplasmin. The HEXB is also known as Hexosaminidase subunit beta. The SERPINA4 is also known as Serpin Family A member 4 or Kallistatin. The VWF is also known as von Willebrand factor. Sequences of the proteins or the genes encoding the same may be identified from a known database such as Ensembl Genome Browser or BLAST.

As used herein, the term “diagnosis” refers to identifying the presence or characteristics of a pathological condition, and may include identifying whether or not a disease related to pre-eclampsia has developed, or identifying the possibility of developing the disease.

The term “measuring level” may include measuring the level of expression or activation of a marker for a disease related to pre-eclampsia in a biological sample, in order to diagnose a disease related to pre-eclampsia. The marker may be metabolites, genetic materials such as DNA or RNA, or a protein expressed from the genetic material or a peptide which is a fragment thereof.

The term “pre-eclampsia” refers to hypertensive disease associated with pregnancy. In addition, the pre-eclampsia may be a disease in which proteinuria occurs with hypertension. Specifically, the pre-eclampsia may be a concept that includes chronic hypertension, gestational hypertension, and pre-eclampsia and eclampsia, which are more advanced forms of the condition. Symptoms of the pre-eclampsia include headache, edema, nausea, vomiting, foamy urine, and the like.

The chronic hypertension means hypertension that occurred before pregnancy or before 20 weeks of pregnancy. The gestational hypertension refers to hypertension that newly occurs after 20 weeks of pregnancy and is normalized within 12 weeks of childbirth. The “normal blood pressure” means systolic blood pressure of less than 140 mmHg and diastolic blood pressure of less than 90 mmHg. The pre-eclampsia refers to a disease, in which hypertension and proteinuria occur at the same time. The eclampsia refers to a disease, in which hypertension, proteinuria, and seizure occur at the same time.

In an embodiment, the formulation for measuring a level of the protein or the fragment thereof may include an antibody or an antigen-binding fragment thereof that specifically binds to the protein or the fragment thereof.

The formulation for measuring a level of the protein or the fragment thereof may be an oligopeptide, a monoclonal antibody, a polyclonal antibody, a chimeric antibody, a ligand, PNA or an aptamer. In addition, the formulation for measuring a level of the protein or fragment thereof may be substituted by a formulation for measuring a level of a metabolite that express the protein or the fragment thereof, or a genetic material such as DNA or RNA.

The antibody is a term known in the related art, and refers to a specific protein molecule directed to an antigenic site. An antibody may be produced by cloning each gene into an expression vector according to a conventional method to obtain a protein encoded by the marker gene, and from the obtained protein by a conventional method. Forms of the antibody may include a monoclonal antibody, a polyclonal antibody, or a chimeric antibody, and optionally, a special antibody such as a humanized antibody.

The aptamer is a single-stranded oligonucleotide, and refers to a nucleic acid molecule having binding activity to a given target molecule. The aptamer may have various three-dimensional structures depending on its nucleotide sequence, and may have high affinity for a specific substance, as in an antigen-antibody reaction. The aptamer may inhibit activity of a given target molecule by binding to the given target molecule.

In an embodiment, the pre-eclampsia may be at least one selected from chronic hypertension, gestational hypertension, pre-eclampsia, eclampsia and complex pre-eclampsia.

Another aspect provides a kit for diagnosing pre-eclampsia comprising the composition.

The terms “pre-eclampsia”, “diagnosis”, etc., are as described above.

In an embodiment, the kit may be a kit for diagnosing pre-eclampsia, which is an enzyme-linked immunosorbent assay (ELISA) kit, a protein chip kit, a rapid kit, or a multiple reaction monitoring (MRM) kit.

The kit may be a protein chip kit for measuring a level of the protein encoded by the gene. The kit may include a substrate, an appropriate buffer solution, a secondary antibody labeled with a chromogenic enzyme or a fluorescent substance, a chromogenic substrate, etc. for immunological detection of the antibody. The substrate may be a nitrocellulose membrane, a 96-well plate synthesized from a polyvinyl resin, a 96-well plate synthesized from a polystyrene resin, or a slide glass made of glass. The chromogenic enzyme may be a peroxidase or an alkaline phosphatase. The fluorescent substance may be FITC or RITC. The chromogenic substrate may be 2,2′-azino-bis(3-ethylbenzothiazoline-6-sulfonic acid) (BTS), 0-phenylenediamine (OPD), or tetramethyl benzidine (TMB).

Still another aspect provides a method of providing information on diagnosis of pre-eclampsia, the method comprising: (a) measuring an expression level of at least one protein selected from the group consisting of ANXA3, A2M, APOB, PZP, FETUB, FNI, LCN2, APOM, QSOXI, TGOLN2, FIO, SERPINAII, PRG2, SHBG, TNC, HBD, AGT, CP, HEXB, SERPINA4, and VWF, or a fragment thereof in a biological sample isolated from a subject; (b) comparing the expression level of the measured protein or the fragment thereof with an expression level of the protein or the fragment thereof in a control group sample.

The terms “pre-eclampsia”, “diagnosis”, “kit”, etc., are as described above.

The term “subject” refers to a subject intended to identify whether or not a pre-eclampsia has developed, to identify the possibility of developing the disease, or to predict the risk of developing the disease. The subject may be a vertebrate, specifically, mammals, amphibians, reptiles, birds, etc., more specifically, mammals, for example, human (Homo Sapiens).

The term, “biological sample” may include whole blood, serum, plasma, saliva, urine, sputum, lymphatic fluid, cells, tissues, etc. isolated from the subject, and preferably may be a sample secreted from a living body, more specifically, nasal discharge, saliva, urine, sputum, or lymphatic fluid isolated from the subject.

The term “control group” may refer to a subject that is not a pre-eclampsia patient.

In an embodiment, the method of providing information on diagnosis of pre-eclampsia may further comprise: determining the subject as pre-eclampsia, when the expression level of the at least one protein selected from the group consisting of ANXA3, A2M, APOB, PZP, FETUB, FNI, LCN2, APOM, QSOXI, TGOLN2, FIO, SERPINAII, PRG2, SHBG and TNC or the fragment thereof, which is measured from the biological sample isolated from the subject in step (b), is increased compared to the control group.

In an embodiment, the method of providing information on diagnosis of pre-eclampsia may further comprise: determining the subject as pre-eclampsia, when the expression level of the at least one protein selected from the group consisting of HBD, AGT, CP, HEXB, SERPINA4, and VWF, or the fragment thereof, measured from the biological sample isolated from the subject in step (b), is decreased compared to the control group.

In an embodiment, in the method of providing information on diagnosis of pre-eclampsia, the expression level of the protein or the fragment thereof may be measured by any one selected from the group consisting of western blotting, enzyme linked immunosorbent assay (ELISA), radioimmunoassay (RIA), radial immunodiffusion, Ouchterlony immunodiffusion, rocket immunoelectrophoresis, immunohistochemical staining, immunoprecipitation assay, complement fixation assay, immunofluorescence, immunochromatography, fluorescenceactivated cell sorting (FACS) analysis, and protein chip technology assay.

According to an example, the subject diagnosed with pre-eclampsia may have significantly higher expression levels of ANXA3, A2M, APOB, PZP, FETUB, FNI, LCN2, APOM, QSOXI, TGOLN2, FIO, SERPINAII, PRG2, SHBG and TNC proteins or fragments thereof measured from a biological sample compared to a control group.

According to an example, the subject diagnosed with pre-eclampsia may have significantly lower expression levels of HBD, AGT, CP, HEXB, SERPINA4, and VWF proteins or fragments thereof measured from a biological sample compared to a control group.

Still another aspect provides a kit for use in diagnosing pre-eclampsia comprising the composition.

Still another aspect provides a use of the formulation for measuring an expression level of at least one protein selected from the group consisting of ANXA3, A2M, APOB, PZP, FETUB, FNI, LCN2, APOM, QSOXI, TGOLN2, FIO, SERPINAII, PRG2, SHBG, TNC, HBD, AGT, CP, HEXB, SERPINA4, and VWF, or the fragment thereof, for diagnosing pre-eclampsia.

Still another aspect provides a method of diagnosing pre-eclampsia comprising: (a) measuring an expression level of at least one protein selected from the group consisting of ANXA3, A2M, APOB, PZP, FETUB, FNI, LCN2, APOM, QSOXI, TGOLN2, FIO, SERPINAII, PRG2, SHBG, TNC, HBD, AGT, CP, HEXB, SERPINA4, and VWF, or the fragment thereof in a biological sample isolated from a subject; (b) comparing the expression level of the measured protein or the fragment thereof with an expression level of the protein or the fragment thereof in a control group sample.

### Advantageous Effects of Disclosure

The composition for diagnosing pre-eclampsia, the method of diagnosing pre-eclampsia using the same, and the method of providing information on diagnosis of pre-eclampsia may be used to accurately and sensitively diagnose the subject's pre-eclampsia.

### BRIEF DESCRIPTION OF DRAWINGS

FIGS. 1A to 1C are results showing up-regulation of expression of 15 proteins ANXA3, A2M, APOB, PZP, FETUB, FNI, LCN2, APOM, QSOXI, TGOLN2, FIO, SERPINAII, PRG2, SHBG and TNC in subjects with pre-eclampsia.

FIG. 2 is results showing up-regulation of expression of 6 proteins of HBD, AGT, CP, HEXB, SERPINA4, and VWF in subjects with pre-eclampsia.

### MODE OF DISCLOSURE

Hereinafter, the present disclosure will be described in more detail through examples. However, these examples are intended to illustrate the present disclosure, and the scope of the present disclosure is not limited to these examples.

### Example 1: Isolation of Protein from Patient's Blood Sample and Hydrolysis into Peptides

The patient selection criteria are as follows. 25 pregnant women with hypertension (≥140/90 mmHg) accompanied by proteinuria (≥300 mg/day) after 20 weeks of pregnancy, the representative symptom of pre-eclampsia, were selected as the disease group, and 29 pregnant women who delivered normally at the end of the pregnancy without clinically specific symptoms for both the fetus and mother were selected as the normal group.

The process of collecting a blood sample, a biological sample from the pregnant women, was as follows. The pregnant woman's blood collected in a 10 cc EDTA tube was subjected to a first centrifugation process (8000 rpm, 15 minutes), and then the isolated supernatant (plasma) was again subjected to a second centrifugation process (13,000 rpm, 10 minutes), and finally, plasma to be used in the experiment was obtained.

Protein isolation and peptide experiments were carried out as follows. 14 high-abundant proteins leading to reduced sensitivity of LC-MS/MS analysis (albumin, [0075] IgG, antitrypsin, IgA, transferrin, haptoglobin, fibrinogen, alpha2-macroglobulin, alpha1-acid glycoprotein, IgM, apolipoprotein Al, apolipoprotein All, complement C3, transthyretin) were removed by using the IGY Column (P/N SEP030-1KT, Sigma). After removal, only proteins less than 5% of the total blood proteins were analyzed.

For the multiple reaction monitoring (MRM), an assay method using a mass spectrometer, a process of cutting proteins into peptide units was required, and fragmentation was carried out by using an enzyme (trypsin). 50 μl of 6 M urea buffer (0.1 M DTT, 50 mM ABC) was added to the remaining 50 μg of protein after removing the abundant proteins, and incubated at 37° C. for 1 hour. The 50 μl sample was treated with 5 μl of 0.5 M IAA and reacted in the dark for 30 minutes. After the reaction, the urea concentration was made less than 1 M by using 400 μl of 50 mM ABC, and then 2 μg of trypsin was added and incubated at 37° C. for 12 hours. After incubation, salts were removed by using pierce c-18 spin columns (P/N: 89870, Theromo) to remove salts used in the reaction and to obtain only peptides.

### Example 2: Identification of Biomarker by Using Multiple Reaction Monitoring (MRM)

Verification of biomarker candidates was carried out as follows. The MRM transition of the candidate group was selected for verification of the biomarker candidates. For the selection of MRM transition, SRM Atlas (www.srmatlas.org) with MS-based verification data for all peptides was used. In this regard, the selection criteria are as follows.

1. Exclude peptides containing methionine (M) and histidine (H).

2. Exclude peptides including a miscleavage

3. Exclude peptides having Arginine (R) or Lysine (K) in the sequence before protein (p).

4. Peptide length is 5<x<20.

Using the selected candidate transition, a primary target selection was preceded and the suitability of the transition was identified. The transition identification conditions are as follows.

1. Whether 3 types of fragment ions derived from the same precursor ion have the same dissolution time

2. Whether to have the same retention time (RT) in the experiments repeated 3 or more times.

3. Whether signa to noise (S/N)>10 that meets the limit of quantification (LOQ) condition is satisfied

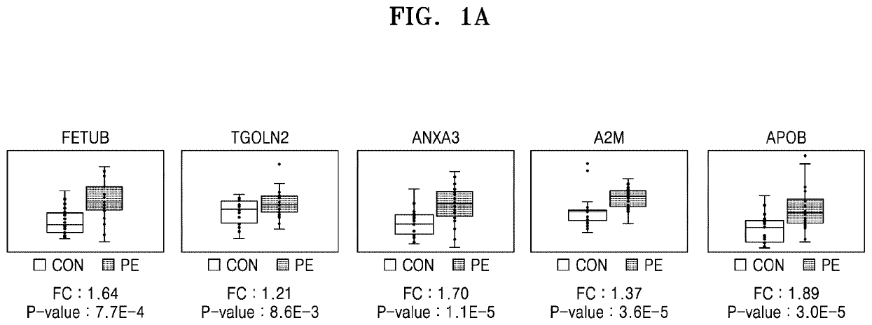

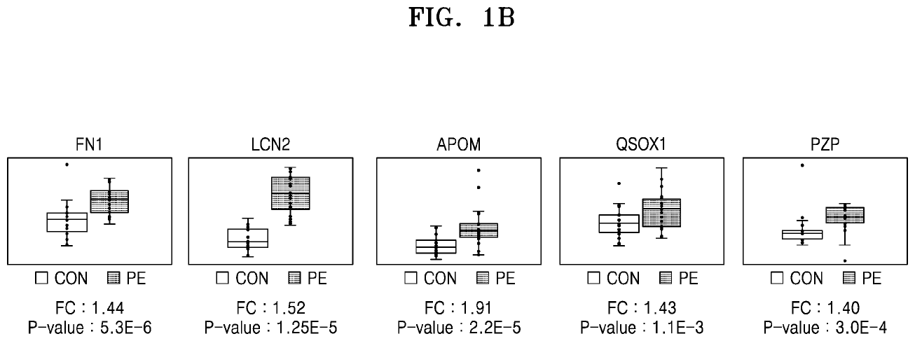

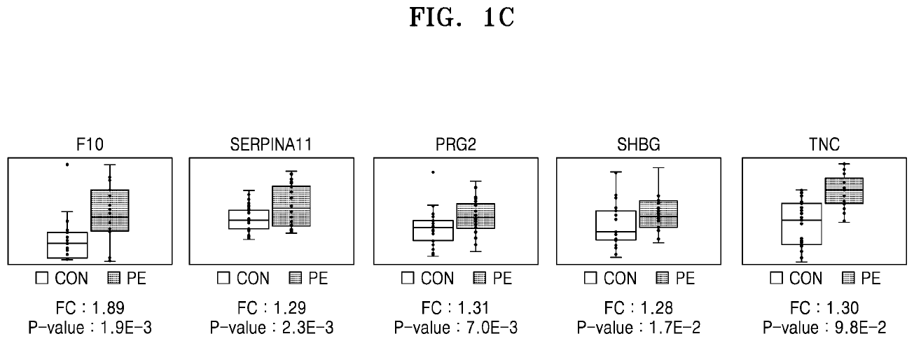

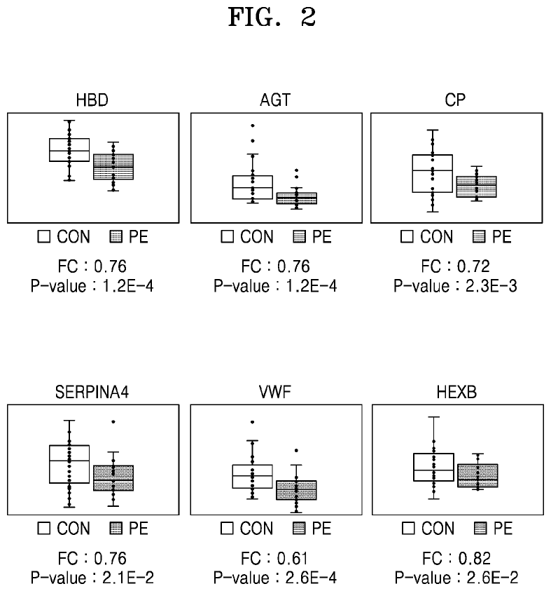

The results of an MRM analysis were obtained through the following process. After selecting a transition that satisfied the above conditions, an MSM analysis was performed on the prepared peptide samples. As shown in FIGS. 1A to 1C and 2, proteins ANXA3, A2M, APOB, PZP, FETUB, FNI, LCN2, APOM, QSOXI, TGOLN2, FIO, SERPINAII, PRG2, SHBG, TNC, HBD, AGT, CP, HEXB, SERPINA4, and VWF were verified as proteins with significantly different expression levels in the blood of pregnant women with pre-eclampsia compared to that of normal pregnant women. 15 proteins showed a significant increase (ANXA3, A2M, APOB, PZP, FETUB, FNI, LCN2, APOM, QSOXI, TGOLN2, FIO, SERPINAII, PRG2, SHBG and TNC) and 6 proteins showed a significant decrease (HBD, AGT, CP, HEXB, SERPINA4, and VWF).

1. A composition for diagnosing pre-eclampsia comprising: a formulation for measuring an expression level of at least one protein selected from the group consisting of ANXA3, A2M, APOB, PZP, FETUB, FNI, LCN2, APOM, QSOXI, TGOLN2, FIO, SERPINAII, PRG2, SHBG, TNC, HBD, AGT, CP, HEXB, SERPINA4, and VWF, or a fragment thereof.

2. The composition for diagnosing pre-eclampsia of claim 1, wherein the formulation for measuring a level of the protein or the fragment thereof is an antibody or an antigen-binding fragment thereof that specifically binds to the protein or the fragment thereof.

3. The composition for diagnosing pre-eclampsia of claim 1, wherein the pre-eclampsia is at least one selected from the group consisting of chronic hypertension, gestational hypertension, pre-eclampsia, eclampsia and complex pre-eclampsia.

4. A kit for diagnosing pre-eclampsia comprising the composition of claim 1.

5. The kit for diagnosing pre-eclampsia of claim 4, wherein the kit is an enzyme-linked immunosorbent assay (ELISA) kit, a protein chip kit, a rapid kit, or a multiple reaction monitoring (MRM) kit.

6. A method of providing information on diagnosis of pre-eclampsia, the method comprising:
(a) measuring an expression level of at least one protein selected from the group consisting of ANXA3, A2M, APOB, PZP, FETUB, FNI, LCN2, APOM, QSOXI, TGOLN2, FIO, SERPINAII, PRG2, SHBG, TNC, HBD, AGT, CP, HEXB, SERPINA4, and VWF, or a fragment thereof in a biological sample isolated from a subject; (b) comparing the expression level of the measured protein or the fragment thereof with an expression level of the protein or the fragment thereof in a control group sample.

7. The method of providing information on diagnosis of pre-eclampsia of claim 6, further comprising: determining the subject as pre-eclampsia, when the expression level of the at least one protein selected from the group consisting of ANXA3, A2M, APOB, PZP, FETUB, FNI, LCN2, APOM, QSOXI, TGOLN2, FIO, SERPINAII, PRG2, SHBG, and TNC, or the fragment thereof, measured from the biological sample isolated from the subject in step (b), is increased compared to the control group.

8. The method of providing information on diagnosis of pre-eclampsia of claim 6, further comprising: determining the subject as pre-eclampsia, when the expression level of the at least one protein selected from the group consisting of HBD, AGT, CP, HEXB, SERPINA4, and VWF, or the fragment thereof, measured from the biological sample isolated from the subject in step (b), is decreased compared to the control group.

9. The method of providing information on diagnosis of pre-eclampsia of claim 6, wherein the expression level of the protein is measured by any one selected from the group consisting of western blotting, enzyme linked immunosorbent assay (ELISA), radioimmunoassay (RIA), radial immunodiffusion, Ouchterlony immunodiffusion, rocket immunoelectrophoresis, immunohistochemical staining, immunoprecipitation assay, complement fixation assay, immunofluorescence, immunochromatography, fluorescenceactivated cell sorting (FACS) analysis, and protein chip technology assay.
